(19) United States
(12) Patent Application Publication
(10) Pub. No.: US 2021/0347816 A1
(43) Pub. Date: Nov. 11, 2021

(54) METHOD FOR AFFINITY PURIFICATION

(71) Applicant: BAC IP B.V., Naarden (NL)

(72) Inventors: Wilhelmus Josephus Johanna HERMANS, Oud-Gastel (NL); Mark Ronald TEN HAAFT, Delft (NL); Anja OVERWEEL, Oegstgeest (NL)

(21) Appl. No.: 17/239,195

(22) Filed: Apr. 23, 2021

Related U.S. Application Data
(63) Continuation of application No. 15/616,781, filed on Jun. 7, 2017, now abandoned.
Application No. 15/616,781 is a continuation of application No. 14/820,446, filed on Aug. 6, 2015, now abandoned.
Application No. 14/820,446 is a continuation of application No. 11/792,145, filed on Mar. 5, 2008, now abandoned.

(30) Foreign Application Priority Data
Dec. 2, 2004 (EP) 040778282.3

(51) Int. Cl. C07K 1/22; A61K 39/44; C07K 16/42

(52) U.S. Cl. CPC C07K 1/22; A61K 39/44; C07K 16/42; C07K 2317/22; C07K 2317/92; C07K 2317/31; C07K 2317/34; C07K 2317/51; C07K 2317/565; C07K 2317/569

(57) ABSTRACT

The invention relates to a method of immunoaffinity purification which comprises the use of a binding agent which binds to an epitope that is present at least twice on the target molecule. In another embodiment the method uses at least two different binding agents, each binding to different epitopes on the target molecule.

### CROSS-REFERENCE TO RELATED APPLICATIONS

This application is a continuation of U.S. application No. continuation of U.S. application Ser. No. 15/616,781 filed Jun. 7, 2017, which is a continuation of U.S. application Ser. No. 14/820,446 filed Aug. 6, 2015, now abandoned, which is a continuation of U.S. application Ser. No. 11/792,145 filed Mar. 5, 2008, now abandoned, which is a 371 of International Application No. PCT/USNL2005/000829 filed Dec. 2, 2005, and claims priority to European Application No. 04078282.3 filed Dec. 2, 2004, which disclosures are incorporated by reference in their entirety.

### SEQUENCE LISTING

The instant application contains a Sequence Listing which has been filed electronically in ASCII format and is hereby incorporated by reference in its entirety. Said ASCII copy, created on Oct. 23, 2020, is named LT00826CON2_SL.txt and is 39,359 bytes in size.

### FIELD OF THE INVENTION

The invention relates to a method for affinity purification and to a binding agent for use in that process.

### BACKGROUND OF THE INVENTION

The use of binding agents in affinity purification is known from EP-A-434317. This document discloses that it is possible to immobilise small specific binding agents on a solid phase carrier. The binding agents are especially composed of corresponding VH and VL domain proteins, held together by their natural interaction. On immobilization these small binding agents maintain affinity for their target ligand. An example of the technology disclosed is the immobilisation of an anti-lysozyme Fv on agarose and its use as an immunoadsorbent. The immunoadsorbent comprising immobilized anti-lysozyme-Fv was packed in a column and on this column a mixture of lysozyme and other protein was loaded. The column was washed to remove non-bound protein and subsequently bound protein was eluted.

In this type of immunoaffinity systems the binding strength, specificity and capacity of the immunoadsorbent are important parameters.

Binding strength and specificity refer to the binding between the binding agent and the target. Especially the binding agent is an antibody or fragment thereof, and the binding is highly specific for the target. Desirably, specific binding to the target is maximized and non-specific binding is minimised. Binding with only very limited specificity is underlying the commercial purification systems that are based on protein A and protein L. These are known to bind to a wide range of immunoglobulin molecules such as immunoglobulins from human, mouse and rat.

The strength of the binding should be balanced between good binding such that the column is easily loaded with target material and no target material is eluted prior to elution, and the wish to use mild elution conditions to avoid unnecessary harm to the target molecules. Very stringent elution conditions are undesired because they may lead to denaturation, fragmentation or other defects of the target material. Furthermore mild conditions are beneficial to the column material and increase the lifetime thereof.

Capacity of the immunoadsorbent indicates the amount of target material that can be bound by the immunoadsorbent material under binding conditions. Once maximum capacity of the immunoadsorbent material has been obtained, the further loading of target will not result in binding but will cause leakage of target material in the eluent. This either results in a loss of yield in protein purification or requires the inclusion of (repeated) further process steps.

In EP-A-434317 some of these objectives are addressed for example by use of variable domains having reduced affinity for antigen, leading to elution or desorption under milder conditions. Non-specific binding is reduced by reducing the size of the binding agent down to the minimum required for binding.

Although these measures to some extent may improve the capacity, specificity and affinity characteristics of the immunoadsorbent, there is still a desire for further improved immunoadsorbent materials.

### SUMMARY OF THE INVENTION

We have surprisingly found that an immunoadsorbent material comprising a specific binding agent, which is preferably an antibody or fragment thereof, that binds to at least two epitopes on a target, has the desired high affinity while elution can still be done under mild conditions. When used as a material for column chromatography it was found to lead to high capacity of the column.

Therefore the invention relates to a method for purification of a target molecule by immunoaffinity, which comprises use of an immunoadsorbent material comprising a binding agent, with binding affinity for an epitope that is present at least twice in the target molecule.

In another aspect the invention relates to a method for purification of a target molecule by immunoaffinity, which comprises use of an immunoadsorbent material comprising at least two different binding agents, wherein the binding agents comprise a binding agent with binding affinity for an epitope (one) on the target molecule and a binding agent with binding affinity for a different epitope (two) on the target molecule.

In a further aspect the invention relates to an immunoadsorbent material comprising a specific binding agent, having binding affinity for an epitope that is present at least twice on a target molecule.

### DESCRIPTION OF THE INVENTION

Target molecule is defined as a molecule that should bind to a binding agent such as an antibody. Examples of target molecules are proteins that require purification, proteins that are to be identified.

Binding affinity (KD) is defined in the context of specific binding and is represented by an affinity of at least 1*10⁶ M⁻¹. Preferably the binding affinity (measured on the isolated binding agent and target), is at least 1*10⁷ M⁻¹, more preferred at least 1*10⁸ M⁻¹.

Immunoadsorbent material is herein understood to mean the combination of a carrier and a binding agent that is immobilized on the carrier. The carrier may be any material that may be used to for immobilization of a binding agent. Suitable examples are matrix materials, to entrap the binding agent, cell surfaces on which the binding agent is displayed and polymers that can be covalently linked to the binding agent. The person skilled in the art of affinity chromatography is well aware of suitable carriers such as e.g. SEPHAROSE™ resin. The binding agent is preferably immobilized onto the carrier by a covalent link.

An epitope is defined as the portion of the target molecule that is bound by the binding agent. If the binding agent is an antibody, it is the portion of a target molecule that triggers an immunological response on immunization of a species with this molecule. Generally it is the site of the target molecule where binding to an antibody takes place.

The epitope is preferably present naturally in the target molecule. Optionally the epitope(s) is/are a sequence that has been artificially included in the target molecule. Optionally a multitude of the same or different epitopes is included in the target molecule to facilitate its purification and detection.

The binding agent is a component that specifically binds to the target molecule with the desired binding affinity. The binding agent preferably is a mono-specific binding agent. A composition comprising a mono-specific binding agent, such as the immunoadsorbant materials of the present invention, is understood to mean a composition having a homogeneous population of the binding agent. It follows that the mono-specific binding agent is specific for a single epitope or ligand. It is however expressly included in the invention that the immunoadsorbant material may comprises more than one type of mono-specific binding agent, each consisting of a homogeneous population. Usually, however, in the context of the present invention, an immunoadsorbant material will not comprise more than 4, 6, 8, 10 or 20 different mono-specific binding agents. It is highly preferred that the binding agent is an antibody or fragment thereof. In that case the mono-specific binding agent will thus be a monoclonal antibody or a fragment thereof, which may be obtained from a hybridoma or expressed from a cloned coding sequence. The term mono-specific binding agent as used herein thus excludes polyclonal antibodies and antisera. An example of a suitable fragment that may be used is the binding part of the antibody, referred to as CDR, (complementary determining region). Such fragments may suitably be inserted on a natural or synthetic scaffold molecule such as a synthetic peptide.

The invention relates to a method of immunoaffinity purification, which comprises the use of a binding agent, which binds to a target molecule in at least two positions. This is referred to as multivalent binding. In one embodiment the binding agent binds to an epitope that is present at least twice on the target molecule. In another embodiment the method uses at least two different binding agents, each binding to different epitopes on the target molecule.

Accordingly the invention relates to a method for purification of a target molecule by immunoaffinity, which comprises use of an immunoadsorbent material comprising a binding agent, which comprises either
a) A binding agent with binding affinity for an epitope that is present at least twice in the target molecule
b) or comprise one binding agent with binding affinity for an epitope (one) on the target molecule and a different binding agent with binding affinity for a different epitope (two) on the target molecule.
   Below preferred further steps of this method are presented.

In an alternative embodiment, the invention relates to a method for purification of a target molecule by immunoaffinity which comprises the use of a binding agent according to (a) or (b) above, in combination with at least one binding agent that shows monovalent binding to the target molecule. Such multiple binding agent combinations are especially suitable for use if the individual binding agent has a binding affinity below 1*10⁸ M⁻¹, for the target.

Optionally the method of the invention is a combination of (a) and (b) above.

In a preferred embodiment the invention relates to a method for purification of a target molecule, wherein the method comprises the step of binding the target molecule to an immunoadsorbent material that comprises at least one mono-specific binding agent, and wherein the binding agent has affinity for at least two epitopes on the target molecule that are spatially separated.

Preferably, the spatial separation of the at least two epitopes on the target molecule is such that binding of a first epitope on the target molecule to a binding agent does not substantially interfere with binding of a second or further epitope to a binding agent. This means that binding of a first epitope on the target molecule to a binding agent does not substantially reduce or “block” binding of a second or further epitope to a binding agent. The absence of substantial blocking is herein understood to mean that the binding of a first epitope on the target molecule to a binding agent does not reduce the binding of a second or further epitope to a binding agent by more than 50, 40, 30, 20, 10 or 5%. The absence of substantial blocking may be determined in standard cross-blocking assays (see e.g. “Using Antibodies” E. Harlow and D. Lane, Cold Spring Harbor Laboratory Press NY, 1999) or as described in Example 5 herein.

The spatial separation of the at least two epitopes on the target molecule is thus preferably such that at least two epitopes on the target molecule are available for binding to a binding agent on the immunoadsorbent material. The spatial separation of the at least two epitopes is thus such that it results in multivalent binding. The multivalent binding results in a significantly lower dissociation rate (k-diss) of the target molecule for the immunoadsorbent material comprising the binding agent(s) and significantly higher K_{D} values. Preferably the presence of at least two spatially separated epitopes on the target molecule produces at least a 5-, 10-, 50-, 100-, 500- or 1000-fold increase in affinity or K_{D} value for the immunoadsorbant material comprising a binding agent as compared to the affinity or K_{D} value of a target molecule comprising only a single copy of the same epitope. It is herein understood that 120 fM is a higher affinity or K_{D} value than e.g. 6.3 nM. The presence of at least two spatially separated epitopes on the target molecule produces thus induces avidity for the immunoadsorbant material comprising a binding agent as compared to a target molecule comprising only a single copy of the epitope. Avidity is herein understood to refer to the strength of binding of a target molecule with multiple binding sites by a larger complex of binding agents, i.e. the strength of binding of multivalent binding. Affinity, on the other hand refers to simple monovalent receptor ligand systems.

Thus in a preferred embodiment, the avidity of the immunoadsorbant material for the target molecule is at least 5-, 10-, 50-, 100-, 500- or 1000-fold higher than the lowest affinity of a binding agent for an individual epitope on the target molecule. The lowest affinity herein refers to the situation wherein the immunoadsorbant material comprises more than one type of binding agent for immunologically distinct epitopes on the target molecule and wherein each individual type of binding agent may have a different affinity for its epitope. In the situation wherein a single type of binding agent binds an epitope that is repeated on the target molecule the lowest affinity is the affinity of the binding agent for a test molecule comprising only a single copy of the epitope.

The spatial separation of the at least two epitopes on the target molecule is further thus preferably such that the dynamic binding capacity of the immunoadsorbant material comprising a binding agent for the target molecule is increase by at least 10, 20, 50, 100 or 200% as compared to the dynamic binding capacity of the material for to a target molecule comprising only a single copy of the epitope. The dynamic binding capacity (DBC) is herein defined as the elution peak area devised by the total peak area (flowthrough+elution) and this multiplied with the amount of target molecule (see Example 3).

Another important feature of the current invention is that although multivalent binding of the target molecule to the immunoadsorbant material leads to high binding affinities or avidity for the target molecule, the release of said molecules can still be obtained at mild elution conditions. Thus in a preferred embodiment, the avidity of an immunoadsorbant material comprising a binding agent for the target molecule is at least 1*10⁹ 1*10¹⁰ M⁻¹, 1*10¹¹ M⁻¹, or 1*10¹² M⁻¹. Preferably, at the same at least 90% of the target molecule can be eluted from the immunoadsorbant material at a pH of 2.5, 2.75, 3.0, 3.25 3.5 or higher. However, preferably the individual affinities of a binding agent for the an epitope on the target molecule are less than 1*10¹⁰ M⁻¹, 1*10¹¹ M⁻¹, or 1*10¹² M⁻¹.

The above defined features of binding of the target molecule to the immunoadsorbant material may be achieved when the at least two epitopes on the target molecule are spatially separated by at least 10, 20, 25, 30,40, 50 or 60 Angstrom. In the methods of the invention, the at least two epitopes on the target molecule may be at least two immunologically distinct epitopes. In which case at least two distinct binding agents are required in the immunoadsorbant material, preferably one for each immunologically distinct epitope.

Alternatively, the methods of the invention, the at least two epitopes on the target molecule may be at least two immunologically identical epitopes that are repeated on the target molecule. In which case only a single type of binding agent is required in the immunoadsorbant material. Target molecules with repetitions of epitopes are e.g. homo-multimeric protein such as e.g. immunoglobulins comprising 2 light and 2 heavy chains.

The method preferably comprises a step of selecting the binding agent. This selection is preferably done under conditions mimicking the loading and elution conditions to find a binding agent that binds with sufficient affinity to bind the target molecule on loading and which can also be eluted easily.

In one embodiment of the invention, the binding agent should have binding affinity for an epitope that occurs at least twice on the target molecule. By this selection binding agents, especially antibodies or fragments thereof, are selected, which could provide multivalent binding to a target molecule. In use two individual binding agents, especially antibody molecules, may bind to different epitopes of the same target molecule, leading to multivalent binding of the target molecule. This multivalency increases the binding affinity and the capacity of the immunoadsorbent with surprisingly high levels. Without wishing to be bound by any theory, it is believed that the multivalent binding is essential to achieve the high affinity, or rather avidity, the high dynamic binding capacity and mild elution which are objectives of the current invention.

Examples of such antibodies or fragments thereof are antibodies that specifically recognize an epitope that is present on the kappa or lambda light chain of human antibodies. In this example the human antibody is the target molecule. Human antibodies comprise two kappa light chains which comprise the epitopes.

In another embodiment of the invention in the selection step, at least two binding agents are selected, comprising one binding agent with binding affinity for an epitope (one) on the target molecule and one binding agent with binding affinity for a different epitope (two) on the target molecule. In use the adsorbent material will comprise these two different binding agents, especially antibodies or fragments thereof, that may simultaneously bind to the same target molecule, again leading to multivalent binding of the target molecule.

As mentioned above the multivalent binding of target molecule, i.e. each target molecule is bound to the immunoadsorbent material via at least two binding agent-epitope bonds, is a preferred aspect of the current invention.

It will be appreciated that the multivalent binding will take place optimally if the 3 dimensional structure of the target molecule is such that it allows this multiple binding. In practice this means that a certain distance is preferably present between the two epitopes on the target molecule. The suitability of the target molecule for multivalent binding can be derived from its 3D crystal structure.

The selection of the antibody or fragment is preferably followed by its production in larger quantities. Suitable production systems include Saccharomyces Cerevisiae or other yeasts, mould or bacterial expression systems.

Preferably this is followed by a step wherein the binding agent is brought into contact with immunoadsorbent material. The immunoadsorbent and the binding agent may be linked covalently or via other interactions. Preferably the binding agent is covalently coupled to an immunoadsorbent material. There are many known protocols for immobilizing proteins or fragments on adsorbent material.

It will be appreciated that where reference is made to immunoadsorbent comprising “one” or “a” binding agent, this is meant to also refer to the situation where an immunoadsorbent material is loaded with many individual binding agent molecules.

Examples of suitable immunoadsorbent material include porous solid phase carrier materials such as agarose, polystyrene, controlled pore glass, cellulose, dextrans, kieselguhr, synthetic polymers such as SEPHAROSE™ resin, porous amorphous silica. The carrier materials may be in any suitable format such as particles, powders, sheets, beads, filters and the like. Further specifications of suitable carrier materials are for example disclosed in EP-A-434317.

Methods are available for immobilizing ligands quickly, easily and safely through a chosen functional group. The correct choice of coupling method depends on the substance to immobilized. For example the following commercially known derivatives of SEPHAROSE™ resin allow the convenient immobilization of proteins thereon: CnBr-activated SEPHAROSE™ resin 4B enables ligands containing primary amino groups to be rapidly immobilized by a spontaneous reaction.

AH SEPHAROSE™ resin 4B and CH SEPHAROSE™ resin 4B both have a six-carbon long spacer arm and permit coupling via carboxyl and amino groups respectively. Flexible spacers are suitable for use in situations where the flexibility of the target molecules is limited or where 3-dimensional structure of the target requires some flexibility of the binding agent to allow optimal binding.

Activated CH SEPHAROSE™ resin 4B provides a six-carbon spacer arm and an active ester for spontaneous coupling via amino groups.

These are only a few examples of suitable immobilisation routes.

Optionally the immunoadsorbent material is put into a column to facilitate easy chromatographic separations.

In a preferred next step the immunoadsorbent material is contacted with a composition comprising the target. Often the composition will be an aqueous composition comprising many other proteins besides the target that is to be purified. The conditions of this contact step are such that binding of the binding agent, to the target molecule occurs.

Preferably in this step a loading buffer having pH around 6.5 to 8 is used. A suitable buffer is PBS buffer.

It is preferred that the loaded material is rinsed until the non specific binders have eluted.

Desorption of the target molecule is the next step. This is preferably done by changing the conditions such that the antibody or fragment no longer binds the target molecule.

Elution may be achieved by changing the conditions with respect to pH, salt, temperature or any other suitable measure. A preferred elution method for desorption is elution with a buffer having a pH below 3.

More specifically the invention relates to a method for the purification of a target molecule by immunoaffinity comprising the steps of:

a) selecting an antibody or fragment thereof, that binds to an epitope that is present at least twice on the target molecule;

b) binding the antibody or fragment thereof to immunoadsorbent material;

c) loading the immunoadsorbent material with a composition comprising the target molecule, preferably under conditions where multivalent binding of the binding agent to the target molecule takes place;

d) washing the loaded immunoadsorbent to remove non specific binders; and,

e) eluting the target molecule by applying elution conditions.

The binding agent may be derived from any suitable source organism or may be prepared synthetically or by genetically modified organisms. It is important that the fragment retains binding affinity as defined above in the context of this invention.

Examples of suitable fragments are Fab fragments, Fv fragments.

In a preferred embodiment, the binding agents are antibodies, more preferred such antibodies or fragments thereof are derived from antibodies naturally devoid of light chains. Such antibodies are for example obtained by immunisation of llama's or sharks and purification of the antibodies thus generated. These antibodies comprise heavy chains only and are devoid of light chains. The advantage of use of these antibodies is that they are exceptionally stable even at higher temperatures, small and are easily produced in host organisms such as Saccharomyces cerevisiae. These antibodies are described in more detail in EP-A-656946.

More generally speaking, the binding agent preferably comprises immunoglobulin-derived variable domain that comprises a complete antigen binding site for an epitope on the target molecule in a single polypeptide chain. Such agents thus specifically include but are not limited to:

1) antibodies obtainable from camelids and sharks that consist of only heavy chains and that are naturally devoid of light chains;

2) variable domains of the antibodies defined in 1), usually referred to as VHH domains;

3) engineered forms of the antibodies defined in 1) such as e.g. “camelidised” antibodies in which frame work sequences of a camelid (or shark) VHH domain are grafted with CDRs obtained from other sources;
4) engineered forms of immunoglobulin-like variable domains in which frame works sequences from a variety of immunoglobulin-like molecules are combined with CDRs specific for a given target molecule as e.g. described in WO 04/108749.

A definition of CDR and framework sequences is given herein below. It is further understood that the frame work amino acid sequence of an immunoglobulin-derived variable domain that comprises a complete antigen binding site for an epitope on the target molecule in a single polypeptide chain preferably has at least 50, 55, 60, 65, 70, 75, 80, 85 or 90% amino acid identity with the frame work amino acid sequence of any one of SEQ ID No's 1-33. Preferably the immunoglobulin-derived variable domain has an affinity for the epitope as defined above herein.

A preferred binding agent is a single domain antibody fragment derived from a camelid antibody. Derived from a camelid antibody herein only means that the frame work amino acid sequences of the fragment are as defined above but that the fragment may be designed and synthesised rather than directly obtained from or formed in a camelid organism.

We have found that the immunoadsorbent material of the invention, comprising an binding agent that binds specifically to the kappa light chain of human antibodies, show very good affinity, capacity and need only mild elution conditions.

Therefore in a specific embodiment, the invention relates to an immunoabsorbent material comprising a binding agent that has binding affinity for the kappa light chain of human antibodies.

The invention further relates to the use of such material in the purification of human antibodies.

In one embodiment the invention relates to a binding agent that is selected from the group of kappa light chain binding VHH molecules selected from SEQ ID No's 1-15, or a binding agent comprising an immunoglobulin-derived variable domain comprising a Complementarity Determining Region (CDR) 1, 2, and/or 3 exhibiting at least 80, 85, 90, 95, 98% amino acid identity with the CDRs of the VHH molecules of SEQ ID No's 1-15. Preferably, an immunoglobulin-derived variable domains have an affinity for the kappa light chain of human antibodies that is at least 10⁶ M⁻¹, 10⁷ M⁻¹, or 10⁸ M⁻¹. The CDRs 1, 2, and 3 may each individually exhibit the above defined identities or the CDRs may collectively exhibit the above defined identities.

The Complementarity Determining Regions (CDR) 1, 2, and/or 3 of VHH molecules are herein defined as depicted in FIGS. 3 and 4. More generally, CDRs of camelid variable heavy chain domains are herein defined as described by Vu et al. (1997, Mol Immunol. 34 (16-17): 1121-31). The VHH sequences other than the CDR sequences in SEQ ID No's 1-33 or as defined by Vu et al. (1997, supra) are herein defined as VHH framework sequences.

In another more specific embodiment the invention relates to an immunoadsorbent material comprising an antibody or active fragment thereof having the amino acid sequence specified below as Sequence ID 1.

Sequence ID 1:
QVQLQESGGGLVQPGGSLRLSCAASGRTISRYAMSWFRQAPGKEREFVA
VARRSGDGAFYADSVQGRFTVSRDDAKNTVYLQMNSLKPEDTAVYYCAI
DSDTFYSGSYDYWGQGTQVTVSS

In another embodiment the invention relates to an antibody or fragment having binding affinity for a human antibody, wherein the sequences comprise the sequence according to sequence ID no 1 or are essentially homologous thereto. In the context of the invention essentially homologous means that the homologous molecule shows the desired binding affinity of at least 10⁶ M⁻¹. Examples of homologous sequences are sequences that have been modified in the framework but not in the antigen binding part of the antibody according to sequence ID 1.

In yet another embodiment the invention relates to a binding agent that is selected from the group of lambda light chain binding VHH molecules selected from SEQ ID No's 16-33, or a binding agent comprising an immunoglobulin-derived variable domain comprising Complementarity Determining Regions (CDR) 1, 2, and/or 3 exhibiting at least 80, 85, 90, 95, 98% amino acid identity with the CDRs of the VHH molecules of SEQ ID No's 16-33. Preferably, an immunoglobulin-derived variable domains have an affinity for the lambda light chain of human antibodies that is at least 10⁶ M⁻¹, 10⁷ M⁻¹, or 10⁸ M⁻¹. The CDRs 1, 2, and 3 may each individually exhibit the above defined identities or the CDRs may collectively exhibit the above defined identities.

In yet a further embodiment, the immunoadsorbant material comprises at least two different binding agents each binding an immunologically distinct epitope of a human IgG Fc domain, whereby the immunologically distinct epitopes of the human IgG Fc domain, are spatially separated as herein defined above.

In again another embodiment, the immunoadsorbant material comprises at least two different binding agents each binding an immunologically distinct epitope of human serum albumin, whereby the immunologically distinct epitopes of the albumin are spatially separated as herein defined above.

In another embodiment the current invention provides methods and immuno-adsorbent materials that are highly suitable for the adsorption or isolation and/or purification of immunoglobulins from crude preparations, in particular of human immunoglobulins of the types IgM, IgA, IgE and most in particular of the IgG isotypes.

Immuno adsorbent materials comprise as immunoglobulin binding agents heavy chains naturally devoid of a light chain, preferably camelid VHH's, that are specific for an epitope that is present at least twice on the target immunoglobulin, which may be identical or different targets.

In one preferred embodiment, the target epitope may comprise an epitope present on a light chain of the kappa or lambda isotypes, which targets are always present in duplo on an intact immunoglobulin.

In another embodiment it may be an epitope that is present twice on the Fc part of the immunoglobulin, or two different epitopes that are present on the Fc part and simultaneously accessible for interaction with the VHH binding molecule.

In yet another embodiment, the immuno adsorbent materials may comprise combinations of kappa or lambda binding antibodies with Fc binding antibodies, preferably camelid VHH antibodies, normally devoid of a light chain.

The use of immunoglobulin binding agents specific for epitopes present at least twice on the target immunoglobulin results in a surprising decrease in the dissociation rate of 10 fold to over 1000 fold in some cases, as demonstrated in the examples section for kappa binding antibodies. The decrease in the dissociation rate is useful for isolation and purification processes, as the dynamic binding capacity of immuno-adsorbent materials (or retention rate) is dramatically increased by the decreased dissociation. Secondly, the immunoglobulins can still be eluted under mild conditions, which is crucial for retaining their structural integrity and antigen binding properties.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The invention is illustrated by the following examples.

### DESCRIPTION OF THE FIGURES

FIG. 1 Alignments of human kappa and human lambda light chain binding VHHs and consensus for the CDR′ regions.

FIG. 2 Sensorgrams of BIACORE™ surface plasmon resonance system affinity measurements; Fab—and IgG binding curves in BIACORE™ surface plasmon resonance system on a VHH Hu-kappa-1 coated sensor chip (A; binding and dissociation, B; dissociation only, indicating the effect on the dissociation rate (kdiss) induced by avidity.

FIG. 3 CDR amino acid sequences of human kappa light chain binding VHH molecules.

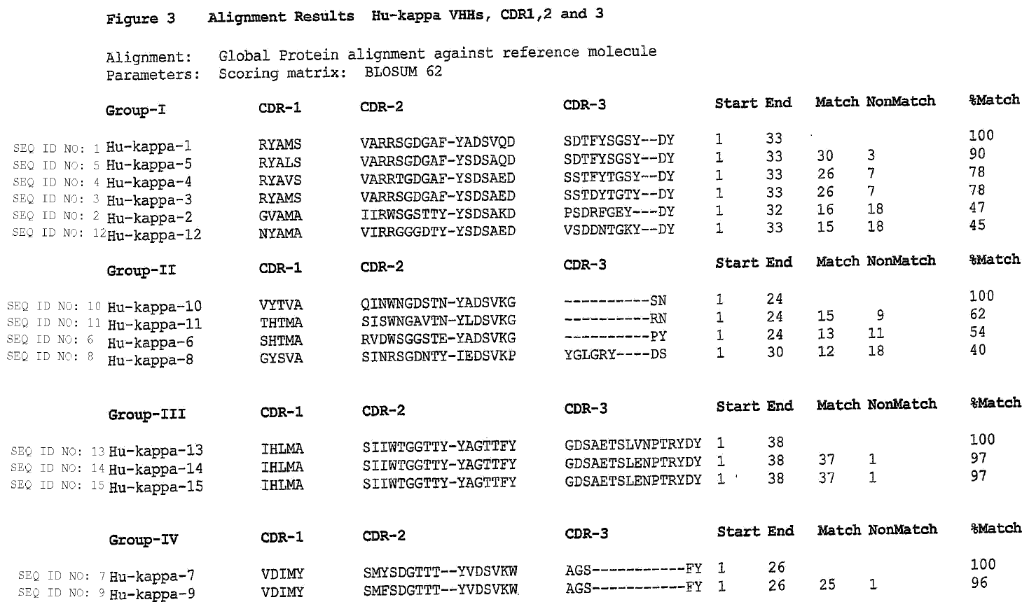

FIG. 4 CDR amino acid sequences of human lambda light chain binding VHH molecules.

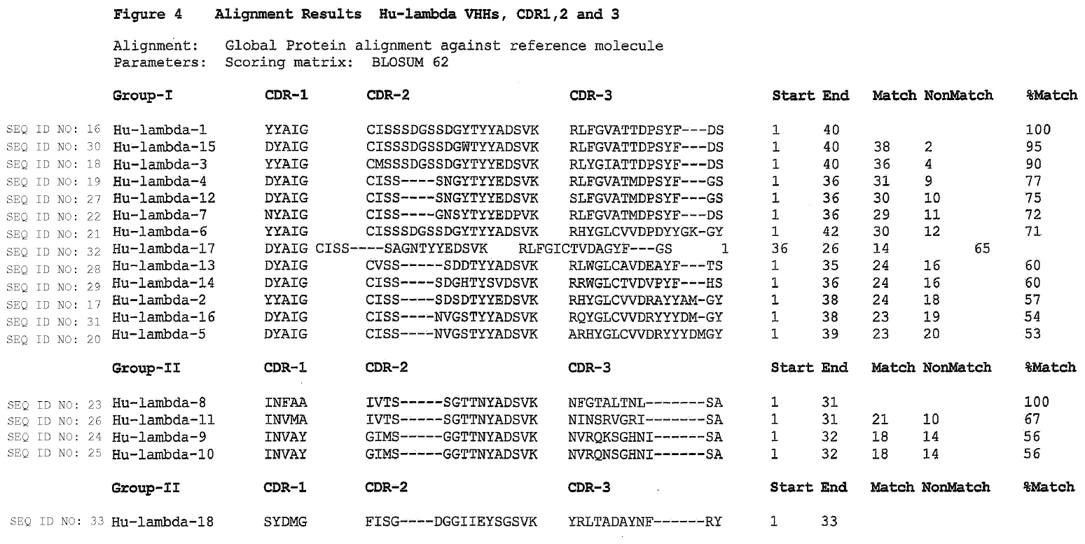

### EXAMPLES

### Example 1: Generation of Llama VHH Ligands Against Kappa Light Chains of Human Antibodies

The following protocol is taken as an example of how specific VHH fragments can be isolated, cloned, expressed, then coupled onto the desired matrix.

Although the particular VHH fragments described in this example were derived from an immune repertoire, they also could have been selected from a non-immunized VHH library (see EP1051493, Unilever) or a synthetic/semi-synthetic non-immunized VHH library (see WO00/43507, Unilever).

A llama was immunized with polyclonal IgM prepared from human serum by precipitation and gel filtration techniques and diluted in phosphate buffered saline pH 7.4 (PBS). To increase the specificity of the immune response the llama was boosted several times following the initial immunization (days 0, 28 and 49) with 250 μg of the antigen mentioned above in specol (ID-DLO, Lelystad, The Netherlands) (Boersma et al., 1992). A heparin blood sample of about 150 ml was taken 6 days after the last immunization. Peripheral blood cells were obtained via a FICOLL-PAQUE™ density gradient medium centrifugation. From about 2×10⁸ lymphocytes the total RNA was isolated essentially as described by Chomczynnski and Sacchi (1987). DNA encoding specific VHH fragments can then be isolated using similar methods to those described in WO 94/04678 (Casterman et al) and ligated into e.g. an episomal Saccharomyces cerevisiae expression vector as previously described by Frenken et. al. (2000). From these VHH expression libraries, VHH fragments with the desired antigen binding specificity (kappa or lambda light chains of human antibodies) can be selected by direct screening of culture supernatants from individual S. cerevisiae colonies (Frenken et. al. 2000).

Alternatively selection methods based on display techniques like phage-display and yeast display can be used to isolate anti kappa light chain VHH producing clones from immune repertoires.

For screening NUNC™ MAXISORP™ binding plates were coated with human antibody antigens and subsequently blocked with 4% (w/v) milkpowder in PBS. Bound VHH fragments were detected by either a mouse anti-His mAb in combination with a polyclonal goat-anti-mouse-HRP conjugate (Bio-Rad, 172-1011) or a polyclonal rabbit anti-llama-VHH serum in combination with a polyclonal swine-anti-rabbit IgG-HPO conjugate (Dako, P217). Initial screening was performed on NUNC™ MAXISORP™ plates either coated with a human IgG1 monoclonal antibody that possessed a kappa light chain or a human IgG1 mab that possessed a lambda light chain. Llama VHH fragments that showed binding to IgG1 kappa only were further tested in ELISA on different human monoclonal antibodies (e.g. IgG, IgA, IgM) to confirm binding specificity towards the human kappa light chain. During the screening process, additional VHH fragments could be identified that showed binding specificity towards the human lambda light chain. The binding specificity of one of the anti human kappa VHH fragments (VHH Hu-kappa-1) as determined by ELISA is given in Table 1 and herein compared with two other VHH fragments that specifically bind to the Fc portion of human IgG antibodies (VHHs Hu-Fc-1 and Hu-Fc-2, respectively). These VHH fragments were obtained from an immune VHH library originating from a llama immunized with human Fc fragments prepared from polyclonal IgG from human serum. As demonstrated in Table 1, VHH Hu-kappa-1 recognizes an epitope that is present on kappa light chains of human antibodies and hence binds to an epitope that is present twice in the target molecule in the case of e.g. IgG, IgA and IgE antibodies or ten times in the case of IgM antibodies. Both VHH fragments Hu-Fc-1 and Hu-Fc-2 show to be specific for an epitope that is present on the Fc domain of all 4 human IgG subclasses, said epitope being present only once in the IgG target molecule.

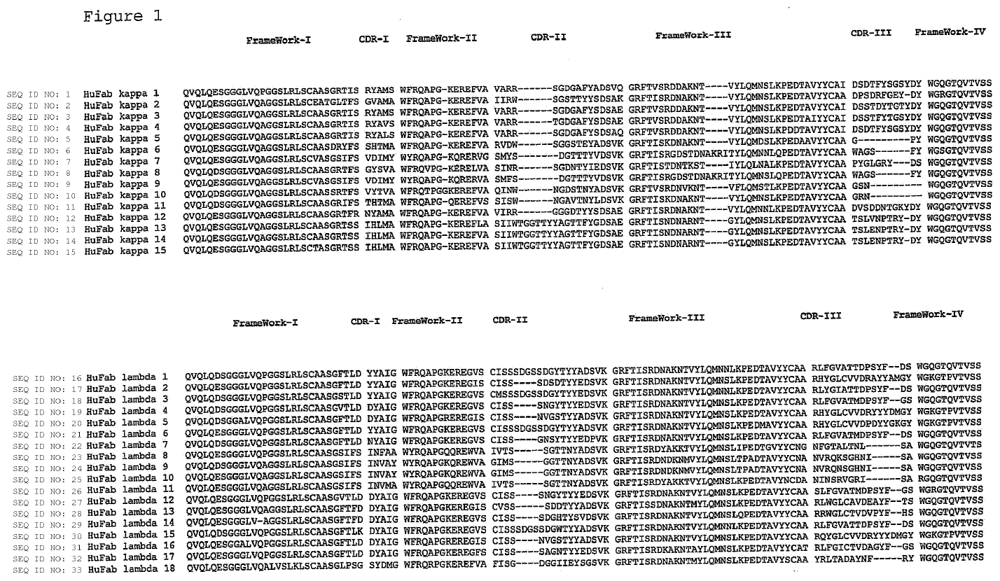

The sequence of these antibodies is presented in sequence ID no's 1 to 15 for the kappa light chain binding VHHs, no's 16 to 31 for the lambda binding VHHs. The consensus for both kappa and lambda binders is displayed in FIG. 1.

TABLE 1
Binding specificity of anti human kappa light chain and
anti human IgG Fc VHH fragments as determined in ELISA.

| Antibody: | | VHH fragment: | | |
|---|---|---|---|---|
| Species | Isotype/Subclass | Hu-kappa | Hu-Fc-1 | Hu-Fc-2 |
| Human | IgG₁ lambda | − | + | + |
| | IgG₂ lambda | − | + | + |
| | IgG₃ lambda | − | + | + |
| | IgG₄ lambda | − | + | + |
| | IgG₁ kappa | + | + | + |
| | IgG₂ kappa | + | + | + |
| | IgG₃ kappa | + | + | + |
| | IgG₄ kappa | + | + | + |
| | IgG, Fab fragments | + | − | − |
| | IgG, Fc fragments | − | + | + |
| | IgM (polyclonal) | + | − | − |
| | IgA (polyclonal) | + | − | − |
| Bovine | IgG (polyclonal) | − | − | − |
| Mouse | IgG (polyclonal) | − | − | − |

### Antibody Production and Immobilized Metal Affinity Chromatography (IMAC) Purification

The VHH antibody fragments were produced at 1-Litre or 10-Litre (shake flasks) fermentation scale using a genetically modified strain of Saccharomyces cerevisiae and purified using ion exchange chromatography on SP SEPHAROSE™ fast flow resin (Amersham Biosciences). The purified antibodies were dialysed against three changes of PBS buffer, pH 7.4 or the required coupling buffer, 48 hours at 4° C., using 3.5 kDa cut-off tubing (SPECTRA/PRO™ 3 dialysis tubing; Spectrum Medical Industries). The concentrations of the purified samples were determined by OD₂₈₀. All purified samples were stored at −20° C. when not in use.

### Example 2: Affinity Measurements

Binding affinity constants of VHH fragments Hu-kappa-1, Hu-Fc-1 and Hu-Fc-2 were determined using surface plasmon resonance analysis (SPR) on a BIACORE™ 3000 surface plasmon resonance system. For this purpose, purified VHH fragments were immobilised onto the surface of a CM5 sensor chip and subsequently incubated with different concentrations of human Fab and/or human IgG antibodies in HBS-EP buffer (0.01 M HEPES, pH7,4; 0.15 M NaCl; 3 mM EDTA; 0.005% Surfactant P20). Binding was allowed for 3 minutes at 30 μl/min followed by a dissociation step of 15 minutes at 30 μl/min. Binding curves were fitted according to a 1:1 Langmuir binding model using BIACORE™ surface plasmon resonance system software. An overview of the calculated affinity data is given in Table 2. With regard to the anti Hu-kappa-1 fragment the effect of avidity is clearly demonstrated by major differences in dissociation rates between Fab—and IgG molecules. Since the anti Hu-kappa-1 fragment is immobilised onto the surface of a sensor chip, said surface can interact with two identical epitopes present on one IgG molecule simultaneously. Compared to the monovalent interaction with human Fab fragments, the dissociation rate (kdiss) for IgG is significantly lower (>1000 fold) resulting in a KD value for IgG of about 120 fM compared to 6.3 nM for Fab fragments. The latter value is in the same range as the KD values for the two anti Hu-Fc VHHs (6.4 and 2.2 nM for IgG, respectively) indicating monovalent interactions.

TABLE 2
BIACORE ™ surface plasmon resonance affinity data of anti Hu-
kappa-1 and anti Hu-Fc VHHs (VHHs immobilised on sensor chip).

| | | k ass | k diss | KA | KD | |
|---|---|---|---|---|---|---|
| VHH | Antigen | (1/Ms) | (1/s) | (1/M) | (M) | Avidity |
| Hu-kappa-1 | Hu-Fab | 1.24 × 10⁵ | 7.78 × 10⁻⁴ | 1.60 × 10⁸ | 6.27 × 10⁻⁹ | No |
| Hu-kappa-1 | Hu-IgG | 4.18 × 10⁵ | 5.29 × 10⁻⁸ | 7.90 × 10¹² | 1.27 × 10⁻¹³ | Yes |
| Hu-Fc-1 | Hu-IgG | 2.15 × 10⁵ | 1.39 × 10⁻³ | 1.55 × 10⁸ | 6.44 × 10⁻⁹ | No |
| Hu-Fc-2 | Hu-IgG | 8.30 × 10⁵ | 1.78 × 10⁻³ | 4.66 × 10⁸ | 2.15 × 10⁻⁹ | No |

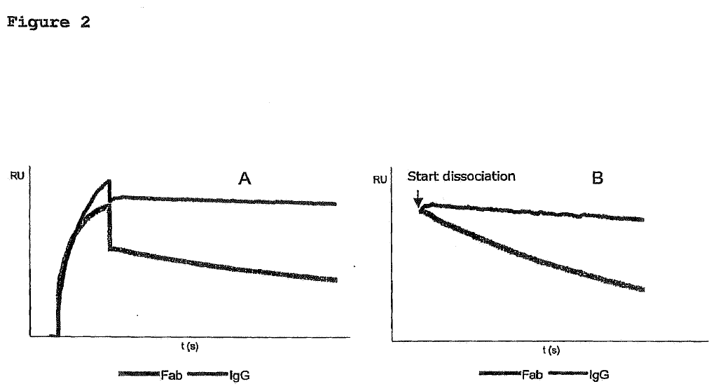

This effect on the dissociation rate (kdiss) induced by avidity is further illustrated by FIG. 2, which compares Fab—and IgG BIACORE™ surface plasmon resonance system binding curves (sensorgrams).

### Example 3: General Materials and Methods for Coupling and Chromatography Testing

### Coupling to N-Hydroxysuccinimide (NHS) Activated Sepharose™ 4 Fast Flow Resin

After purification the antibodies were dialysed to NHS coupling buffer. This buffer contains 0.1 M HEPES pH 8.3. For an optimal coupling efficiency the recommended ratio of volumes coupling solution/NHS SEPHAROSE™ resin is 0.5:1. The antibodies had different concentrations, 0.5-15 mg/ml, the ratio of antibody/NHS SEPHAROSE™ resin of the antibodies varied between 1:1 and 10:1. When a mixture of 2 ligands is immobilised onto the matrix a 1:1 ratio of ligands was used. The following procedure was used for coupling the antibodies to NHS activated SEPHAROSE™ 4 Fast Flow resin (General Electric Healthcare). Subsequently the matrix was washed twice with NHS coupling buffer. The NHS SEPHAROSE™ resin was mixed with the antibody solution and left overnight at 4′C head over head or 1 hour at room temperature. After incubation the gel material was filtered over a sintered glass filter and the non-reacted groups of the gel material were blocked with Tris (0.1 M pH 8.0) 1 hour at room temperature. The coupled medium was washed using alternate low and high pH (3× 10 cv PBS pH 2 and 3× 10 cv PBS pH 7.4). Using the non-bound fraction the coupling efficiency was determined. This is determined by measuring the OD₂₈₀ value of the coupling solution before and after coupling. The coupling efficiency was also determined by looking at the protein pattern on SDS-PAGE of coupling solution before and after coupling.

### Chromatography Experiments

Columns were made of the coupled antibody matrix using HR 5/5 columns (GE health). A column volume of 400 μl was used. All the chromatography experiments were performed on an AKTAEXPLORER™ 100 FPLC system. A two buffer system was used, buffer A1 the loading buffer was PBS pH 7.4, buffer B the elution buffer e.g. PBS with addition of 8 M HCl to yield pH 2.1 or 0.1 M Glycine-HCl at pH 2 or 3. Different programs were used. Protein detection was performed on line by monitoring the signal of OD₂₁₄ and OD₂₈₀. Fractions were collected with a volume of 1 ml. The fractions were neutralized immediately with 20 μl Tris (2 M).

### Determination of the Dynamic Binding Capacity of the Antibody Coupled Matrix

To determine the dynamic binding capacity the column was loaded with the target molecule. A flowrate of 150 cm/h was used. The amount of target molecule in the flowthrough and the elution were calculated by integration of the flowthrough and elution peak area. The dynamic capacity is the elution peak area devised by the total peak area (flowthrough+elution) and this multiplied with the amount of target molecule.

### Example 4: Dynamic Binding Capacities of Different VHH Matrices for Human IgG

VHH fragments (ligands) Hu-kappa-1, Hu-Fc-1 and Hu-Fc-2 were immobilised onto NHS SEPHAROSE™ resin as described earlier. Ligand densities were 20 mg of ligand per ml matrix. The dynamic binding capacity was determined using procedures as described above. The column was loaded with an amount of Human IgG higher than the expected dynamic binding capacity. Elution was performed using 0.1M Glycine buffers with pH values between 2 and 4.

As can be seen from Table 3, the highest dynamic binding capacity (DBC) for human IgG is found for the Hu-kappa-1 matrix and is almost a 2-fold higher compared to both Hu-Fc-1—and Hu-Fc-2 matrices. Affinity measurements showed that the binding affinity (KD) of this antibody for human IgG molecules (containing 2 identical kappa light chains) is about 120 fM. This is significantly higher than the actual binding affinity of Hu-kappa-1 for its epitope present on the kappa light chains as determined with monovalent binding Fab fragments (6.3 nM). The latter figure is more in the range of both tested Human Fc specific VHH fragments and generally known compositions in the prior art. This shows that the use of immuno-adsorbent materials according to the invention leads to increased binding capacity compared to systems that do not include the feature of multivalent binding.

Another important feature of the current invention is that although multivalent binding ligands leads to high binding affinities for the target molecule, the release of said molecules can still be obtained at mild elution conditions. As demonstrated in Table 3 the optimal pH of elution for the Hu-Fc-2 matrix is lower (pH 2) compared to the Hu-kappa-1—and the Hu-Fc-1 matrix (both pH 3). The binding strength of the Hu-Fc-2 fragment for its epitope on human IgG is almost 3-fold higher compared to Hu-Fc-1 and Hu-kappa-1 (2.2 nM versus 6.4 nM and 6.3 nM, respectively). Although multivalent binding of the Hu-kappa-1 matrix increases the dynamic binding capacity for human IgG compared to e.g. the Hu-Fc-1 matrix, this avidity feature clearly does not affect conditions to obtain efficient release of bound IgG. Said conditions being comparable to those for systems that do not include the feature of multivalent binding (e.g. Hu-Fc-1 matrix).

The results as mentioned in this example are measured with a low bed height and short residence time. Higher bed heights will increase residence time and the dynamic capacity. The dynamic capacity of the described Hu-kappa-1 column at 15 cm bed height and 150 cm/hr linear flow rate is 30 to 40 mg Human IgG/ml matrix. This is about a two-fold higher than the capacity of known systems that do not include the feature of multivalent binding which is specific for the current invention.

The example furthermore demonstrates that the multivalent principle of the current invention enables a unique combination of high binding capacity on one-hand and mild elution conditions on the other hand.

TABLE 3
Comparison of the dynamic binding capacity
(DBC) of anti Human IgG matrices.

| | | | pH of elution | |
|---|---|---|---|---|
| | Ligand | | giving >90% | KD for |
| | Density | DBC (mg | release of | HuIgG |
| Matrix | (mg/ml) | HuIgG/ml) | bound IgG | (M−1) |
| Hu-kappa-1 | 20 | 24 | 3 | 1.27 × 10⁻¹³ |
| Hu-Fc-1 | 20 | 14 | 3 | 6.44 × 10⁻⁹ |
| Hu-Fc-2 | 20 | 15 | 2 | 2.15 × 10⁻⁹ |

### Example 5: Dynamic Binding Capacities of Matrices for Human IgG Comprising VHHs that Bind to Different Epitopes Present in the Target Molecule

This example demonstrates the feature of increased binding capacity induced by multivalent binding using at least two different VHH ligands, each ligand recognizing a different epitope on the target molecule, in this case human IgG antibodies.

For this purpose two anti human IgG Fc ligands (Hu-Fc-1 and Hu-Fc-2) were used, each ligand binding to a different epitope present on the Fc domain of human IgG antibodies as demonstrated by BIACORE™ surface plasmon resonance system binding analysis (see Table 4). In this BIACORE™ surface plasmon resonance system experiment, purified anti human IgG Fc ligands were immobilised onto the surface of a CM5 sensor chip and subsequently incubated with human Fc fragments. This human Fc capturing step was followed by incubation with either VHH ligand Hu-Fc-1 or Hu-Fc-2. Table 4 shows that VHH ligand Hu-Fc-2 can bind to human Fc fragments when captured by immobilised VHH ligand Hu-Fc-1 and vice versa (51 RU and 181 RU, respectively) which demonstrates that each ligand binds to a different epitope present on the Fc domain of human IgG antibodies. No significant binding signals were obtained in this set-up using identical ligand pairs, indicating that each individual ligand binds to an epitope on the Fc domain of human IgG antibodies that is present or available only once.

TABLE 4
Binding signals of anti Hu-Fc VHHs on captured human Fc fragments
in BIACORE ™ surface plasmon resonance system.

| BIACORE ™ surface plasmon resonance system set-up: | | | Binding |
|---|---|---|---|
| Immobilized | Capture target | anti Hu-Fc VHH | Signal |
| VHH | (100 μg/ml) | (100 μg/ml) | (RU) |
| Hu-Fc-1 | Human Fc | Hu-Fc-1 | 6.0 |
| Hu-Fc-1 | Human Fc | Hu-Fc-2 | 51.0 |
| Hu-Fc-2 | Human Fc | Hu-Fc-2 | 9.6 |
| Hu-Fc-2 | Human Fc | Hu-Fc-1 | 181.9 |

This further illustrates that although IgG antibodies consist of two identical heavy—and light chains, ligands directed against antibodies can recognise epitopes that are formed by a combination of identical chains such as present in the Fc domain of said antibodies. Another feature that can occur is that the ligand binds to one part of the heavy chain of the Fc domain in such a way that it prevents binding of another identical ligand due to steric hindrance.

In this respect, light chain domains of antibodies (e.g. anti human kappa light chains) as demonstrated in Example 4, have proven to be ideal epitopes to generate specific ligands against to allow multivalent binding to an epitope that is present twice in the case of antibodies. Said ligands are clearly not affected by this feature of steric hindrance.

Three different batches of anti human IgG Fc matrices were constructed (Hu-Fc-1, Hu-Fc-2 and Hu-Fc-1/2) onto NHS SEPHAROSE™ resin using methods as previously described. For each matrix the final ligand density was 2 mg of ligand per ml matrix. The dynamic binding capacity was determined using the procedure as previously described. The column was loaded with an amount of human IgG higher than the expected dynamic binding capacity. As can be seen from Table 5, the dynamic binding capacity for the mixed matrix Hu-Fc-1/2 is higher than for each individual matrix. The dynamic binding capacity for the mixed matrix (2.45 mg human IgG/ml matrix) is 1.5 times higher than the average value of the individual matrices (1.65 mg human IgG/ml matrix) that at maximum could be expected when no multivalent binding would have occurred on the mixed matrix. Based on the multivalent binding principle of the current invention these results demonstrate that high binding capacity matrices can be obtained using combinations of different ligands, each ligand binding to a different epitope present on the target molecule.

TABLE 5
Comparison of the dynamic binding capacity
(DBC) of mixed anti human IgG matrices.

| | Ligand Density | DBC |
|---|---|---|
| Mixed Matrix | (mg/ml) | (mg HuIgG/ml) |
| Hu-Fc-1 | 2 | 1.95 |
| Hu-Fc-2 | 2 | 1.35 |
| Hu-Fc-1/Hu-Fc-2 | 2 | 2.45 |

### Example 6: Dynamic Binding Capacities of Matrices for Human Serum Albumin Comprising VHHs that Bind to Different Epitopes Present in the Target Molecule

This example demonstrates the feature of increased binding capacity induced by multivalent binding using at least two different VHH ligands, each ligand recognizing a different epitope on the target molecule, in this case human serum albumin.

For this purpose three different anti human serum albumin (HSA) specific VHH ligands were used (HSA-1, HSA-2 and HSA-3). These VHH ligands were obtained from an immune VHH library originating from a llama immunized with human serum albumin. BIACORE™ surface plasmon resonance system binding analysis according to methods as previously described revealed that ligands HSA-2 and 3 bind to the same epitope present on HSA, whereas ligand HSA-1 binds to a different epitope allowing multivalent binding of HSA when VHH ligand HSA-1 is used in combination with either VHH ligand HSA-2 or 3.

Five different batches of anti HSA matrices were constructed (HSA-1, HSA-2, HSA-3, HSA-1/2 and HSA-2/3) onto NHS SEPHAROSE™ resin using methods as previously described. For each matrix the final ligand density was 2 mg of ligand per ml matrix. The dynamic binding capacity was determined using the procedure as previously described. The column was loaded with an amount of HSA higher than the expected dynamic binding capacity. As can be seen from Table 6, the dynamic binding capacity for the mixed matrix HSA-1/2 is higher than for each individual matrix and the mixed matrix HSA-1/3. The dynamic binding capacity of mixed HSA-1/2 matrix is 2.14 mg HSA/ml matrix, which is about 1.5 times higher compared to the expected average value of 1.48 mg HSA/ml matrix when no multivalent binding would have occurred on the mixed matrix ((1.72+1.23)/2=1.48). The dynamic binding capacity of HSA for the mixed matrix HSA-2/3 is 0.83 mg HSA/ml matrix. This is in line with the expected average value of 0.85 mg HSA/ml matrix, since both ligands bind to the same epitope present on HSA and therefore can not induce multivalent binding leading to an increased binding capacity as demonstrated by the current invention.

TABLE 6
Comparison of the dynamic binding capacity
(DBC) of mixed anti HSA matrices.

| | Ligand Density | DBC |
|---|---|---|
| Mixed Matrix | (mg/ml) | (mg HSA/ml) |
| HSA-1 | 2 | 1.72 |
| HSA-2 | 2 | 1.23 |
| HSA-3 | 2 | 0.46 |
| HSA-1/2 | 2 | 2.14 |
| HSA-2/3 | 2 | 0.83 |

### REFERENCES

Boersma, W. J. A., Bogaerts, W. J. C., Bianchi, A. T. J., Claassen, E., 1992. Adjuvant properties of stable water-in-oil emulsions: evaluation of the experience with specol. Res. Immunol. 143, 503-512.
Chomczynnski, P., Sacchi, N., 1987. Single step method of RNA isolation by acid guanidium thiocyanate-phenolchloroform extraction. Anal. Biochem. 162, 156-159.
Frenken G. J., Richard H. J. van der Linden, Pim W. J. J. Hermans, J. Wil Bos a, Robin C. Ruuls, Bernard de Geus, C. Theo Verrips, 2000, Journal of Biotechnology 78, 11-21.

1.-16. (canceled)

17. A variable heavy-heavy (VHH) antigen binding molecule derived from a llama,
wherein the VHH antigen binding molecule has binding specificity for an Fab region of a human immunoglobin molecule and
wherein the framework amino acid sequences of the variable domain has at least 50% amino acid sequence identity with the framework amino acid sequence of at least one of SEQ ID NOs 1 through 33.

18. The VHH antigen binding molecule of claim 17, wherein the VHH antigen binding molecule has binding specificity for the Fc region of a human immunoglobin molecule.

19. The VHH antigen binding molecule of claim 17, wherein the VHH antigen binding molecule has binding specificity for a kappa light chain or a lambda light chains of the human immunoglobin molecule.

20. The VHH antigen binding molecule of claim 17, wherein at least one of the CDR1, CDR2, or CDR3 share at least 90% amino acid sequence identity with the corresponding CDR1, CDR2, or CDR3 of a protein having an amino acid sequence set out in at least one of SEQ ID NOs 1 through 33.

21. An immunoadsorbent material comprising the VHH antigen binding molecule of claim 17 bound to a chromatography matrix.

22. A method for purification of a target molecule, the method comprising:
a) contacting the target molecule with the immunoadsorbent material of claim 21 under conditions suitable to form an immunoadsorbent material bound target molecule-VHH antigen binding molecule complex,
b) washing the immunoadsorbent material bound target molecule-VHH antigen binding molecule complex, and
c) releasing the target molecule from the immunoadsorbent material bound target molecule-VHH antigen binding molecule complex.

23. The method of claim 22, wherein the VHH antigen binding molecule has affinity for at least two epitopes on the target molecule that are spatially separated from each other.

24. The method according to claim 23, wherein the spatial separation of the at least two epitopes on the target molecule is such that binding of a first epitope on the target molecule to the VHH antigen binding molecule does not substantially block binding of a second or further epitope to the VHH antigen binding molecule.

25. A method for purification of a target molecule, wherein the method comprises the step of binding the target molecule to an immunoadsorbent material that comprises at least one mono-specific VHH antigen binding molecule and wherein the VHH antigen binding molecule has affinity for at least two epitopes on the target molecule that are spatially separated.

26. The method of claim 25, wherein the VHH antigen binding molecule is a single polypeptide chain and wherein the framework amino acid sequences of the variable domain have at least 50% amino acid identity with the frame work amino acid sequence of at least one of SEQ ID NOs 1 through 33.

27. The method of claim 25, wherein at least one epitope is present on the light chain of an immunoglobulin.

28. The method of claim 25, wherein the at least two epitopes are epitopes of a human immunoglobulin.

29. The method of claim 25, wherein the epitopes are epitopes of a human immunoglobulin light chain of the kappa or lambda isotype.

30. A method for purification of an immunoglobulin, the method comprising:
a) binding the immunoglobulin to an immunoadsorbent material that comprises at least one mono-specific VHH antigen binding molecule selected from the group consisting of a kappa light chain binding VHH antigen binding molecule comprising an amino acid sequence selected from SEQ ID Nos 1 to 15 or a lambda light chain binding VHH antigen binding molecule comprising an amino acid sequence selected from SEQ ID Nos 16 to 33, and
b) purifying the immunoglobulin.

31. The method of claim 30, wherein the immunoadsorbent material has affinity for at least two spatially separated epitopes on the immunoglobulin.

32. The method of claim 31, wherein at least two of the at least two spatially separated epitopes on the immunoglobulin that are spatially separated by at least 30 angstroms.


SEQUENCE LISTING (37 sequences)

SEQ ID NO: 1 - protein, 121 aa, Lama glama
QVQLQESGGGLVQPGGSLRLSCAASGRTISRYAMSWFRQAPGKEREFVAVARRSGDGAFYADSVQGRFTVSRDDAKNTVYLQMNSLKPEDTAVYYCAIDSDTFYSGSYDYWGQGTQVTVSS

SEQ ID NO: 2 - protein, 120 aa, Lama glama
QVQLQESGGGLVQAGGSLRLSCEATGLTFSGVAMAWFRQAPGKEREFVAIIRWSGSTTYYSDSAKGRFTISRDNAKNTVYLQMNSLKPEDTAVYYCAADPSDRFGEYDYWGRGTQVTVSS

SEQ ID NO: 3 - protein, 121 aa, Lama glama
QVQLQESGGGLVQAGGSLRLSCAASGRTISRYAMSWFRQAPGKEREFVAVARRSGDGAFYSDSAEGRFTVSRDDAKNTVYLQMNSLKPEDTAVYYCAIDSSTDYTGTYDYWGQGTQVTVSS

SEQ ID NO: 4 - protein, 121 aa, Lama glama
QVQLQESGGGLVQAGGSLRLSCAASGRTISRYAVSWFRQAPGKEREFVAVARRTGDGAFYSDSAEGRFTVSRDDAKNTVYLQMNSLKPEDTAIYYCAIDSSTFYTGSYDYWGQGTQVTVSS

SEQ ID NO: 5 - protein, 121 aa, Lama glama
QVQLQESGGGLVQAGGSLRLSCAASGRTISRYALSWFRQAPGKEREFVAVARRSGDGAFYSDSAQGRFTVSRDDAKNTVYLQMNSLKPDDTAVYYCAIDSDTFYSGSYDYWGQGTQVTVSS

SEQ ID NO: 6 - protein, 112 aa, Lama glama
QVQLQESGGGLVQAGGSLRLSCAASDRYFSSHTMAWFRQAPGKEREFVARVDWSGGSTEYADSVKGRFTISKDNAKNTVYLQMDSLKPEDAAVYYCAAGPYWGQGTQVTVSS

SEQ ID NO: 7 - protein, 118 aa, Lama glama
QVQLQESGGGLVQAGGSLRLSCVASGSIFSVDIMYWYRQAPGKQRERVGSMYSDGTTTYVDSVKGRFTISRGDSTDNAKRITYLQMNNLQPEDTAVYYCAAWAGSFYWGSGTQVTVSS

SEQ ID NO: 8 - protein, 118 aa, Lama glama
QVQLQDSGGGLVQAGGSLRLSCAASGRTFSGYSVAWFRQVPGKERELVASINRSGDNTYIEDSVKGRFTISTDNTKSTIYLQLNALKPEDTAVYYCAAPYGLGRYDSWGQGTQVTVSS

SEQ ID NO: 9 - protein, 118 aa, Lama glama
QVQLQESGGGLVQAGGSLRLSCVASGSIFSVDIMYWYRQAPGKQRERVASMFSDGTTTYVDSVKGRFTISRGDSTDNAKRITYLQMNSLQPEDTAVYYCAAWAGSFYWGQGTQVTVSS

SEQ ID NO: 10 - protein, 113 aa, Lama glama
QVQLQDSGGGLVQAGGSLRLSCAASSRTFSVYTVAWFRQTPGGKEREFVAQINWNGDSTNYADSVKGRFTVSRDNVKNTVFLQMSTLKPEDTAVYYCAAGSNWGQGTQVTVSS

SEQ ID NO: 11 - protein, 112 aa, Lama glama
QVQLQDSGGGLVQAGGSLRLSCAASGRIFSTHTMAWFRQAPGQEREFVSSISWNGAVTNYLDSVKGRFTISKDNAKNTVYLQMNSLKPEDTAVYYCAAGRNWGQGTQVTVSS

SEQ ID NO: 12 - protein, 121 aa, Lama glama
QVQLQESGGGLVQAGGSLRLSCAASGRTFRNYAMAWFRQAPGKEREFVAVIRRGGGDTYYSDSAEGRFTISRDNAKNTVYLQMNNLKPEDTAVYYCAADVSDDNTGKYDYWGQGTQVTVSS

SEQ ID NO: 13 - protein, 126 aa, Lama glama
QVQLQESGGGLVQAGGSLRLSCAASGRTSSIHLMAWFRQAPGKEREFLASIIWTGGTTYYAGTTFYGDSAEGRFTISNDNARNTGYLQMNSLKPEDTAVYYCAATSLVNPTRYDYWGQGTQVTVSS

SEQ ID NO: 14 - protein, 126 aa, Lama glama
QVQLQESGGGLVQAGGSLRLSCAASGRTSSIHLMAWFRQAPGKEREFVASIIWTGGTTYYAGTTFYGDSAEGRFTISNDNARNTGYLQMNSLKPEDTAVYYCAATSLENPTRYDYWGQGTQVTVSS

SEQ ID NO: 15 - protein, 126 aa, Lama glama
QVQLQESGGGLVQAGGSLRLSCTASGRTSSIHLMAWFRQAPGKEREFVASIIWTGGTTYYAGTTFYGDSAEGRFTISNDNARNTGYLQMNSLKPEDTAVYYCAATSLENPTRYDYWGQGTQVTVSS

SEQ ID NO: 16 - protein, 128 aa, Lama glama
QVQLQDSGGGLVQPGGSLRLSCAASGFTLDYYAIGWFRQAPGKEREGVSCISSSDGSSDGYTYYADSVKGRFTISRDNAKNTVYLQMNNLKPEDTAVYYCAARLFGVATTDPSYFDSWGQGTQVTVSS

SEQ ID NO: 17 - protein, 126 aa, Lama glama
QVQLQESGGGLVQPGGSLRLSCAASGFTLDYYAIGWFRQAPGKEREGISCISSSDSDTYYEDSVKGRFTISRDNAKNTVYLQMNSLKPEDTAVYYCAARHYGLCVVDRAYYAMGYWGKGTPVTVSS

SEQ ID NO: 18 - protein, 128 aa, Lama glama
QVQLQDSGGGLVQPGGSLRLSCAASGSTLDYYAIGWFRQAPGKEREGVSCMSSSDGSSDGYTYYEDSVKGRFTISRDNAKNTVYLQMNNLKPEDTAVYYCAARLYGIATTDPSYFDSWGQGTQVTVSS

SEQ ID NO: 19 - protein, 124 aa, Lama glama
QVQLQDSGGGLVQPGGSLRLSCAASGVTLDDYAIGWFRQAPGKEREGVSCISSSNGYTYYEDSVKGRFTISRDNAKNTVYLQMNSLKPEDTAVYYCAARLFGVATMDPSYFGSWGQGTQVTVSS

SEQ ID NO: 20 - protein, 126 aa, Lama glama
QVQLQDSGGALVQPGGSLRLSCAASGFTLDDYAIGWFRQAPGREREGISCISSNVGSTYYADSVKGRFTISRDNAKNTVYLQMNSLKPEDTAVYYCAARHYGLCVVDRYYYDMGYWGKGTPVTVSS

SEQ ID NO: 21 - protein, 130 aa, Lama glama
QVQLQESGGGLVQPGGSLRLSCAASGFTLDYYAIGWFRQAPGKEREGVSCISSSDGSSDGYTYYADSVKGRFTISRDNAKNTVYLQMNSLKPEDMAVYYCAARHYGLCVVDPDYYGKGYWGKGTPVTVSS

SEQ ID NO: 22 - protein, 124 aa, Lama glama
QVQLQDSGGGLVQPGGSLRLSCAASGFTLDNYAIGWFRQAPGKEREGVSCISSGNSYTYYEDPVKGRFTISRDNAKNTVYLQMNSLKPEDTAVYYCAARLFGVATMDPSYFDSWGQGTQVTVSS

SEQ ID NO: 23 - protein, 119 aa, Lama glama
QVQLQESGGGLVQPGGSLRLSCAASGSIFSINFAAWYRQAPGQQREWVAIVTSSGTTNYADSVKGRFTISRDYAKKTVYLQMNSLIPEDTGVYYCNGNFGTALTNLSAWGQGTQVTVTS

SEQ ID NO: 24 - protein, 120 aa, Lama glama
QVQLQDSGGGLVQAGGSLRLSCAASGSIFSINVAYWYRQAPGKQREWVAGIMSGGTTNYADSVKGRFTISRDNDKNMVYLQMNSLTPADTAVYYCNANVRQKSGHNISAWGQGTQVTVSS

SEQ ID NO: 25 - protein, 120 aa, Lama glama
QVQLQESGGGLVQAGGSLRLSCAASGSIFSINVAYWYRQAPGKQREWVAGIMSGGTTNYADSVKGRFTISRDNDKNMVYLQMNSLTPADTAVYYCNANVRQNSGHNISAWGQGTQVTVSS

SEQ ID NO: 26 - protein, 119 aa, Lama glama
QVQLQESGGGLVQAGGSLRLSCAASGSIFSINVMAWYRQAPGQQREWVAIVTSSGTTNYADSVKGRFTISRDYAKKTVYLQMNSLKPEDTAVYNCDANINSRVGRISARGQGTQVTVSS

SEQ ID NO: 27 - protein, 124 aa, Lama glama
QVQLQESGGGLVQPGGSLRLSCAASGVTLDDYAIGWFRQAPGKEREGVSCISSSNGYTYYEDSVKGRFTISRDNAKNTVYLQMNSLKPEDTAVYYCAASLFGVATMDPSYFGSWGRGTQVTVSS

SEQ ID NO: 28 - protein, 123 aa, Lama glama
QVQLQESGGGLVQAGGSLRLSCAASGFTFDDYAIGWFRQAPGKEREGISCVSSSDDTYYADSVKGRFTISSDNAKNTMYLQMNSLKPEDTAVYYCAARLWGLCAVDEAYFTSWGQGTQVTVSS

SEQ ID NO: 29 - protein, 123 aa, Lama glama
QVQLQESGGGLVAGGSLRLSCAASGFTFDDYAIGWFRQAPGKEREGVSCISSSDGHTYSVDSVKGRFTISRDNAKNTVYLQMNSLQPEDTAVYYCAARRWGLCTVDVPYFHSWGQGTQVTVSS

SEQ ID NO: 30 - protein, 128 aa, Lama glama
QVQLQDSGGGLVQPGGSLRLSCAASGFTLKDYAIGWFRQAPGKEREGVSCISSSDGSSDGWTYYADSVKGRFTISRDNAKNTVYLQMNNLKPEDTAVYYCAARLFGVATTDPSYFDSWGQGTQVTVSS

SEQ ID NO: 31 - protein, 126 aa, Lama glama
QVQLQESGGALVQPGGSLRLSCAASGFTLDDYAIGWFRQAPGREREGISCISSNVGSTYYADSVKGRFTISRDNAKNTVYLQMNSLKPEDTAVYYCAARQYGLCVVDRYYYDMGYWGKGTPVTVSS

SEQ ID NO: 32 - protein, 124 aa, Lama glama
QVQLQESGGGLVQPGGSLRLSCAASGFTLDDYAIGWFRQAPGKEREGFSCISSSAGNTYYEDSVKGRFTISRDKAKNTAYLQMNSLKPEDTAVYYCATRLFGICTVDAGYFGSWGQGTQVTVSS

SEQ ID NO: 33 - protein, 121 aa, Lama glama
QVQLQESGGGLVQALVSLKLSCAASGLPSGSYDMGWFRQRPGKEREFVAFISGDGGIIEYSGSVKGRFTISRDNAKNTMYLQMNSLKPEDTAVYSCAAYRLTADAYNFRYWGQGTQVTVSS

SEQ ID NO: 34 - protein, 11 aa, Lama glama
  MOD_RES (2)..(2): Asp or Ser
  MOD_RES (4)..(4): Phe or Asp
  MOD_RES (6)..(6): Ser or Thr
  MOD_RES (8)..(8): Ser or Thr
SXTXYXGXYDY

SEQ ID NO: 35 - protein, 5 aa, Lama glama
  MOD_RES (4)..(4): Met, Val, or Leu
RYAXS

SEQ ID NO: 36 - protein, 11 aa, Lama glama
SDTFYSGSYDY

SEQ ID NO: 37 - protein, 121 aa, Lama glama
  MOD_RES (14)..(14): Pro or Ala
  MOD_RES (34)..(34): Met, Val, or Leu
  MOD_RES (54)..(54): Ser or Thr
  MOD_RES (61)..(61): Ala or Ser
  MOD_RES (64)..(64): Val or Ala
  MOD_RES (65)..(65): Gln or Glu
  MOD_RES (89)..(89): Glu or Asp
  MOD_RES (93)..(93): Val or Ile
  MOD_RES (101)..(101): Ser or Asp
  MOD_RES (103)..(103): Phe or Asp
  MOD_RES (105)..(105): Ser or Thr
  MOD_RES (107)..(107): Ser or Thr
QVQLQESGGGLVQXGGSLRLSCAASGRTISRYAXSWFRQAPGKEREFVAVARRXGDGAFYXDSXXGRFTVSRDDAKNTVYLQMNSLKPXDTAXYYCAIDSXTXYXGXYDYWGQGTQVTVSS